(19) United States
(12) Patent Application Publication
(10) Pub. No.: US 2026/0042991 A1
(43) Pub. Date: Feb. 12, 2026

(54) CELL EJECTION APPARATUS, CELL EJECTION METHOD

(71) Applicant: CANON KABUSHIKI KAISHA, Tokyo (JP)

(72) Inventors: TATSUAKI ORIHARA, Tokyo (JP); YOSHINORI ITOH, Tokyo (JP); TSUTOMU SHIRATORI, Tokyo (JP); YUKI MORITA, Tokyo (JP); FUTOSHI HIROSE, Kanagawa (JP); SACHIKO YAMAUCHI, Kanagawa (JP); NOBUYUKI KUWABARA, Tokyo (JP)

(21) Appl. No.: 19/363,993

(22) Filed: Oct. 21, 2025

Related U.S. Application Data
(63) Continuation of application No. PCT/JP2024/016162, filed on Apr. 24, 2024.

(30) Foreign Application Priority Data
Apr. 28, 2023 (JP) 2023-075159
Apr. 22, 2024 (JP) 2024-069368

(51) Int. Cl. C12M 1/26; A23L 13/00; B29C 64/112; B29C 64/209; B29C 64/393; B33Y 40/00; B33Y 50/02; B41J 2/14; B41J 2/175; B41J 2/18; B41J 2/195; C12M 1/12; C12M 1/36; C12M 3/00

(52) U.S. Cl. CPC C12M 33/04; A23L 13/03; B29C 64/112; B29C 64/209; B29C 64/393; B33Y 40/00; B33Y 50/02; B41J 2/14145; B41J 2/1433; B41J 2/175; B41J 2/18; B41J 2/195; C12M 21/08; C12M 25/01; C12M 41/48

(57) ABSTRACT

To provide a cell ejection apparatus capable of reducing the time required to eject a predetermined amount of droplets of a cell suspension containing a cell in an ink jet cell ejection apparatus. A cell ejection apparatus for ejecting a droplet of a cell suspension containing a cell and a liquid by an ink jet system, wherein the cell ejection apparatus comprises a liquid chamber holding the cell suspension, an ejection orifice for ejecting the droplet from the liquid chamber, a supply flow path for supplying the cell suspension to the liquid chamber, a collection flow path for collecting the liquid or the cell suspension from the liquid chamber, and a cell ejection unit for selectively ejecting the droplet from the ejection orifice so that the droplet contains the cell.

### CROSS-REFERENCE TO RELATED APPLICATIONS

This application is a Continuation of International Patent Application No. PCT/JP2024/016162, filed Apr. 24, 2024, which claims the benefit of Japanese Patent Application No. 2023-075159, filed Apr. 28, 2023, and Japanese Patent Application No. 2024-069368, filed Apr. 22, 2024, all of which are hereby incorporated by reference herein in their entirety.

### BACKGROUND

### Field of the Technology

The present disclosure relates to a cell ejection apparatus, a cell ejection method, an apparatus for producing cultured meat, a method for producing cultured meat, an apparatus for producing a tissue, an organ, or a viscus, and a method for producing a tissue, an organ, or a viscus.

### Description of the Related Art

With the development of biotechnology, the importance of bioprinting and cell dispensing, in which cells are arranged in two or three dimensions, has increased. A dispenser system, a laser transfer system, an ink jet system, and the like are known as bioprinting systems. Both methods have advantages and disadvantages, and are often used differently depending on the application. The ink jet system has a high resolution and a high molding speed, but it is difficult to increase the cell concentration of the bioink due to the tendency of nozzle clogging. However, by lowering the cell concentration of the bioink, there were many cases in which no cells were contained in the ejection droplets.

Therefore, in U.S. Patent Application Publication No. 2013/0095469, there is proposed a cell dispensing apparatus which changes the ejection position of the droplet according to whether or not a predetermined requirement such as that no cell is contained in the droplet to be ejected is satisfied.

However, in the cell dispensing apparatus described in U.S. Patent Application Publication No. 2013/0095469, since an extra droplet that does not contain a cell is also ejected, it takes time to eject a predetermined amount of the droplet containing the cell.

### SUMMARY

The present disclosure has been made in view of the above problems, and the present disclosure is directed to provide a cell ejection apparatus capable of reducing the time required for ejecting a predetermined amount of droplets of a cell suspension containing a cell in an ink jet cell ejection apparatus.

The above object is achieved by the cell ejection apparatus according to the present disclosure. The cell ejection apparatus according to the present disclosure is a cell ejection apparatus for ejecting a droplet of a cell suspension containing a cell and a liquid by an ink jet system, wherein the cell ejection apparatus comprises a liquid chamber holding the cell suspension, an ejection orifice for ejecting the droplet from the liquid chamber, a supply flow path for supplying the cell suspension to the liquid chamber, a collection flow path for collecting the liquid or the cell suspension from the liquid chamber, and a cell ejection unit for selectively ejecting the droplet from the ejection orifice so that the droplet contains the cell.

Features of the present disclosure will become apparent from the following description of embodiments with reference to the attached drawings.

### BRIEF DESCRIPTION OF THE DRAWINGS

FIG. 1A is a schematic view showing one embodiment of a cell ejection apparatus according to a first embodiment of the present disclosure.

FIG. 1B is an enlarged view of the cell ejection head 101 of FIG. 1A, viewed obliquely upward.

FIG. 1C is a cross-sectional view of a portion including one ejection orifice 110 in the cell ejection head 101 of FIG. 1B.

FIG. 2A is a schematic view showing an example of ejection control according to the first embodiment of the present disclosure.

FIG. 2B is a schematic view showing another example of ejection control according to the first embodiment of the present disclosure.

FIG. 2C is a schematic view showing yet another example of ejection control according to the first embodiment of the present disclosure.

FIG. 3 is a schematic view showing one embodiment of a cell ejection apparatus according to a second embodiment of the present disclosure.

FIG. 4 is a schematic view showing one embodiment of a cell ejection apparatus according to a third embodiment of the present disclosure.

### DESCRIPTION OF THE EMBODIMENTS

The cell ejection apparatus according to the present disclosure is described in detail below. However, the configuration, structure, materials, dimensions, settings, etc. of the cell ejection apparatus according to the present disclosure should be appropriately changed in accordance with various conditions to which the disclosure is applied, and it is not intended to limit the scope of the present disclosure.

A cell ejection apparatus according to the present disclosure is a cell ejection apparatus for ejecting a droplet of a cell suspension containing a cell and a liquid by an ink jet system, wherein the cell ejection apparatus comprises a liquid chamber holding the cell suspension, an ejection orifice for ejecting the droplet from the liquid chamber, a supply flow path for supplying the cell suspension to the liquid chamber, a collection flow path for collecting the liquid or the cell suspension from the liquid chamber, and a cell ejection unit for selectively ejecting the droplet from the ejection orifice so that the droplet contains the cell.

According to the study by the present inventors, in the cell ejection using the ink jet system, the upper limit of the concentration of the cells that can be stably ejected without causing nozzle clogging in a typical ejection droplet size range of several pl to several 10 pl is about 1×10⁶ cells/ml in terms of the number concentration and about 0.1% in terms of the volume concentration. The typical volume of the cells was about 1 pl, and the probability of cell inclusion in the ejection droplet was as low as 1/1000 to 1/10, and most ejection droplets contained no cells. Therefore, it was necessary to spend a large amount of time to eject a predetermined amount of droplets containing cells. This reduces the efficiency of cell ejection, and in bioprinting, where droplets of cell suspension containing cells are ejected, it causes a reduction in resolution, which is an advantage of the ink jet system. In addition, the fact that droplets that do not contain cells are also ejected unit that the concentration of cells in the ejected droplets is equal to the concentration of cells before ejection, so the bioprinting molded product must also be dilute in cell concentration.

In the present disclosure, a cell suspension containing cells and liquid is flowed from a supply flow path side to a collection flow path side in a cell ejection apparatus, and is selectively ejected so that the droplets contain cells. Therefore, it is possible to reduce the ejection of droplets that do not contain cells, and as a result, the time required to eject a predetermined amount of droplets of the cell suspension containing cells can be reduced. The liquid that has not been ejected and does not contain cells, or the cell suspension after some cells have been ejected, flows into the collection flow path and is collected. Therefore, it is not particularly necessary to provide a mechanism for collecting the liquid containing no cells after ejection. In addition, the amount of liquid containing cells that can be ejected in a unit of time (throughput) can be improved. Further, since the liquid containing cells can always be ejected, it facilitates multi-nozzling wherein a plurality of ejection orifices are provided in the cell ejection apparatus. The cell ejection apparatus of the present disclosure has a plurality of ejection orifices and is suitable for an ink jet cell ejection head having a multi-nozzle integrally formed with the plurality of ejection orifices. The plurality of ejection orifices are preferably arranged in parallel with respect to the flow direction of the cell suspension. Since a plurality of ejection orifices are arranged in parallel, a difference in the pressure of the cell suspension at each ejection orifice is unlikely to occur, and stable ejection is possible even when the flow velocity is particularly high.

The relationship between a direction in which the cell suspension flows in the liquid chamber of the cell ejection apparatus and a direction in which the droplet containing the cell is ejected from the ejection orifice is not particularly limited, but it is preferable that it is substantially perpendicular. If these directions are not substantially perpendicular, the cells may bypass the part where the ejection droplet is generated in the liquid chamber or the ejection orifice, and it is more preferable that these directions are substantially perpendicular. If these directions are substantially perpendicular, the cells can easily pass through the portion where the ejection droplets are generated in the liquid chamber or the ejection orifice, and the droplets containing the cell can easily be selectively ejected from the flow of the cell suspension. The former configuration is sometimes referred to as an edge chute and the latter as a side chute.

The width of the liquid chamber in the direction perpendicular to the flow of the cell suspension in the chamber is not particularly limited, but the narrower width of the liquid chamber is preferably equal to or greater than the cell diameter, and the wider width of the liquid chamber is preferably equal to or less than 5 times the cell diameter, and more preferably equal to or less than 3 times the cell diameter. That is, the liquid chamber has a maximum width W1 and a minimum width W2 in a direction perpendicular to the flow direction of the cell suspension, and when the diameter of the cell is D, it is preferable to satisfy D≤W2≤W1≤5D, and it is more preferable to satisfy D≤W2≤ W1≤3D.

The narrower width of the liquid chamber is larger than the cell diameter, which prevents cells from clogging the liquid chamber or damaging the cells. If the wider width of the liquid chamber is equal to or less than 5 times the cell diameter, the cell can easily pass through the liquid chamber and the ejection orifice area where the ejection droplet is generated, and the cell suspension droplet can be ejected more stably.

The ink jet system is not particularly limited to a thermal ink jet system in which bubble formation of a liquid as heated is used as a driving source, a piezo ink jet system in which piezo is used as a driving source, but is preferably a thermal ink jet system. The thermal inkjet system is suitable for apparatus consisting of side chutes and narrow liquid chambers, because the heating elements can be arranged in a small area within the apparatus.

The cell suspension containing cells may be used without degassing to keep the cells alive, but the dissolved gas may become bubbles, resulting in poor ejection. In the present disclosure, bubbles tend to escape because a constant flow is generated from the supply flow path of the cell suspension to the collection flow path of the liquid or the cell suspension, but in the thermal ink jet system, bubbles can be ejected from the ejection orifice and the displacement per volume is large, so that the poor ejection due to bubbles is less likely to occur.

The configuration of the cell suspension supply flow path and the liquid or cell suspension collection flow path of the cell ejection apparatus is not particularly limited, but it is preferred that each is connected to a different common flow path. If a common flow path exists separately for the supply side of the cell suspension and the collection side of the liquid or cell suspension, it is easier to generate sufficient flow to eject the cells at high throughput by providing a pressure differential between the supply side and the collection side. The common flow path is a common flow path connected to each liquid chamber having a plurality of ejection orifices, and is a common flow path between each liquid chamber on the supply side of the cell suspension and the collection side of the liquid or cell suspension.

A flow rate Q (ml/s) of the cell suspension flowing through the liquid chamber, a number concentration of the cells p (cells/ml) in the cell suspension, and a drive frequency f (Hz) of the cell ejection unit for ejecting the droplets in the ink jet system are not particularly limited, but preferably satisfy Q×p≥f. In an ink jet cell ejection head, the driving timing that enables ejection is digitally controlled. The number of times of driving timing of the cell ejection head per unit time is referred to as a drive frequency. The number of cells supplied into the liquid chamber per unit time can be expressed as Q×p. When this value is small, the cells are supplied only rarely, and the number of cells to be ejected is insufficient even if the cells are ejected at the drive frequency of the cell ejection unit. When Q×p is greater than or equal to f, the cells are fed into the liquid chamber to a sufficient extent to meet the drive frequency, so that the droplets containing the cells can be ejected at a high throughput. Q is preferably 4×10⁻⁵ ml/s or more, and more preferably 4×10⁻⁴ ml/s or more. Further Q is preferably 4×10⁻² ml/s or less, and more preferably 4×10⁻³ ml/s or less. ρ is preferably 10⁵cells/ml or more, and more preferably 10⁶cells/ml or more. Further, ρ is preferably 10⁸cells/ml or less, and more preferably 10⁷cells/ml or less. f is preferably 10 Hz or more, more preferably 100 Hz or more, and even more preferably 1 kHz or more. Further, f is preferably 100 kHz or less. The flow velocity of the cell suspension, obtained by dividing the flow rate of the cell suspension in the liquid chamber by the cross-sectional area of the liquid chamber, is preferably 100 mm/s or more, and more preferably 1 m/s or more. If the flow velocity is within this range, droplets containing cells can be ejected at a high throughput even at a low cell concentration suitable for the ink jet system. The flow velocity is preferably 100 m/s or less, and more preferably 10 m/s or less. In this range, the cell suspension is difficult to be spouted from the ejection orifice. Incidentally, the flow velocity of ink in the liquid chamber in the image recording by the ordinary ink jet system is used at a rate of about 0.1 mm/s or more and 30 mm/s or less to suppress the concentration of pigment contained in ink at the ejection orifice. In other words, it is preferable that the flow velocity of the cell suspension of the present disclosure in the liquid chamber is higher than the flow velocity of the liquid ejection head in the liquid chamber in a conventional ink jet system used for image recording.

The volume of the droplet of the cell suspension ejected from the ejection orifice is not particularly limited, but is preferably 100 times or less of the cell volume. As described above, in the cell ejection using the ink jet system, the concentration of cells capable of stably ejecting cells without nozzle clogging is up to about 0.1% by volume concentration, and it is difficult to exceed about 1%. When the volume of the ejection droplet is 100 times or less of the cell volume, the concentration of the cells contained in the ejection droplet becomes 1% or more when the droplets containing cells are selectively ejected, and the concentration of the cells in the droplet can be increased. From the viewpoint of increasing the cell concentration, it is more preferable that the volume of the droplet of the cell suspension ejected from the ejection orifice is 10 times or less of the cell volume.

More preferably, the volume of the droplet of the cell suspension ejected from the ejection orifice is 6.25 times or less of the cell volume. For positioning cells in bioprinting, it is preferable that the position of the ejected cells does not flow.

As the concentration of cells in the ejection droplet increases, the position of the cells become less fluid. It is generally known that when the concentration of the medium dispersed in the substrate reaches a value called the percolation threshold, an overall connection occurs in the medium. The percolation threshold of a sphere in three dimensions is 16%. When the cell volume is 16% or more of the volume of the ejection droplet, that is, the volume of the ejection droplet is 6.25 times or less of the cell volume, the ejected cells are connected to each other, and the position of the cells is difficult to flow. This effect appears only when the droplet containing no cells can be reduced. When a large number of droplets containing no cells are ejected, the cell suspension after being ejected is difficult to become highly concentrated and therefore easy to flow.

To fix the position of cells in the bioprinting without causing the cells to flow, a liquid that gelates after the ejection droplets land may be used, but this is not always preferable because gelation may use materials that are not originally necessary for the cells themselves, or change the pH or temperature.

The ejection orifice of the cell ejection apparatus is not particularly limited but is preferably formed to narrow from upstream to downstream in the ejection direction of the droplet (that is, a shape in which the flow path cross-sectional area becomes smaller from upstream to downstream in the ejection direction). Cells may be damaged by hydrodynamic action as they pass through the ejection orifice during ejection. When the ejection orifice is formed to narrow from upstream to downstream in the ejection direction, the increase of the flow velocity in the ejection orifice becomes gradual, and the damage of the cell is reduced.

The cell ejection unit for selectively ejecting droplets containing cells in the cell ejection apparatus is not particularly limited, but preferably comprises, at least one of a cell detection unit for detecting a position of the cell in the cell suspension and a cell supply control unit for controlling the position of the cell in the cell suspension; and an ejection control unit for controlling at least one of a timing for ejecting the droplet and a location of the ejection orifice for ejecting the droplet, based on the information on the position of the cell in the cell suspension obtained by the cell detection unit and the information on the position of the cell in the cell suspension controlled by the cell supply control unit. Preferably, the cell ejection unit comprises a cell capture unit for capturing the cells in the cell suspension.

Here, the information on the position of the cell in the cell suspension may be not only the measurement result of the position of the cell but also predicted based on information such as the flow velocity of the cell suspension.

The cell detection unit for detecting information on the position of the cell in the cell suspension is not particularly limited but is preferably optical detection or electrical detection. The optical detection methods are not particularly limited, but cells may be detected by imaging, as in a microscope, or by reflection or scattering, as in an optical sensor.

In order to perform optical detection, at least one of the liquid chamber, the supply flow path, and the collection flow path is preferably at least partially optically transparent. Optical transparency will allow the cell to be detected optically from the outside.

In order to perform electrical detection, it is not particularly limited, but it is preferable to measure impedance. Impedance is the component corresponding to resistance when alternating current is applied. Cells are known to have an impedance different from that of a simple liquid, and cells in a cell suspension can be detected by measuring the impedance. The electrode for electrical detection may be located inside a common flow path, a collection flow path, a liquid chamber, an ejection orifice, or the like in contact with the flow of the cell suspension, or may be located in a portion not in contact with the flow of the cell suspension. A driving electrode, a signal electrode, an electrode of a sensor, and the like used for the ejection of an ink jet system may be diverted.

The cell supply control unit for controlling information on the position of the cell in the cell suspension is not particularly limited, but if the timing of supplying the cell is controlled by a loader or other unit, the information on the position of the cell in the cell suspension can be obtained without specifically detecting the cell.

After information on the position of the cell is acquired by the cell detection unit or the cell supply control unit, the droplet containing the cell is selectively ejected by controlling at least one of the ejection timing and the location of the ejection orifice for ejecting the droplet by the ejection control unit.

Although the ejection control unit is not particularly limited, it is preferable to control the ejection based on both the information on the position of the cell in the liquid and furthermore the image signal. Here, the image signal refers to a signal sent to designate a position where the ejection droplet is disposed. It is more preferable that the ejection control unit controls the ejection based on the information of the cell position, the image signal, and furthermore the allowable deviation amount allowed between the information of the cell position and the image signal.

The following is a simple explanation of the ejection control unit. Cells flow from the supply flow path to the collection flow path. A droplet is ejected aiming at a timing in which a cell is included in the ejection droplet on the basis of information on the position of the cell. This allows selective ejecting of droplets containing cells.

In the case of a single nozzle with one ejection orifice or a cell dispensing application, the position of the cell ejection head may be moved to the position where the droplet is to be ejected, and then the droplet may be ejected at the timing when the cell is included in the ejection droplet. On the other hand, in the case of a multi-nozzle with multiple ejection orifices, a bioprinting application, etc., and the cell ejection head is continuously moved to the target, whether droplets should be ejected at a certain ejection orifice at the position of the cell ejection head at a certain time depends on the image signal. In the ejection orifice where the image signal does not indicate ejection, of course, there is no need to eject even if the cells flow in at the right time. In the ejection orifice where the image signal indicates ejection, the ejection droplet can be disposed faithfully according to the image signal by waiting for the timing when the cells flow and ejecting.

However, waiting for the timing of cells to flow may result in cases where the cell ejection head cannot be moved fast enough. Therefore, a certain amount of allowable deviation should be set between the cell flow timing and the image signal. For example, if the cells do not flow in a timely manner at the timing at which the image signal instructs the ejection, and the cells flow at the timing within the allowable deviation amount or flow to other ejection orifices, the droplets containing the cells are ejected even if the cell flow timing or the image signal at other ejection orifices do not instruct the ejection. Although the arrangement of the ejection droplets is not completely faithful to the image signal, sufficient fidelity can be obtained within the set allowable deviation amount. Alternatively, there is a method of complementing by ejecting from a different path of the cell ejection head movement or from ejection orifices of different rows passing through the same portion. The method of controlling the ejection timing and the ejection orifice is not particularly limited, and various methods can be selected according to conditions and purposes.

In addition to controlling the ejection based on information on the position of the cell, the droplets containing cells can be selectively ejected by capturing the cells and ejecting them. The cell capture unit for capturing the cells is not particularly limited, but is preferably provided in at least one of a liquid chamber, a supply flow path, and a collection flow path. Preferably, the ejection orifice also serves as a cell capture unit. In this case, capturing a cell unit binding the cell to a certain area.

When a cell is captured, since the location of the cell is specified, it is not necessary to obtain information on the position of the cell. If the droplets are ejected while the cells are captured, the droplets containing the cells can be selectively ejected.

By providing the cell capturing unit in the liquid chamber, the supply flow path or the collection flow path, the cells are captured in or near the generation region of the ejection droplets in the liquid chamber or the ejection orifice, and the ejection of the droplets containing the cells is facilitated.

In addition, when the ejection orifice also serves as a cell capture unit, the cells are captured in or near the generation region of the ejection droplets in the liquid chamber or the ejection orifice, and the ejection of the droplets containing the cells is facilitated. When a liquid flows from the supply flow path to the collection flow path, a slow flow or a stagnant flow may occur at the ejection orifice. Because of this flow, cells may be captured in the ejection orifice. If the length of the ejection orifice is short, cells can easily pass through the ejection orifice, but if it is too short, cells cannot be captured. To facilitate cell passage through the ejection orifice, the length of the ejection orifice is not particularly limited, but preferably not more than 5 times the cell diameter, and more preferably not more than the cell diameter. To facilitate cell capture in the ejection orifice, the length of the ejection orifice is not particularly limited, but preferably not less than the cell radius. If the length of the ejection orifice is not less than the cell radius, the center of gravity of the cell will be located in the ejection orifice, and the flow of liquid in the liquid chamber will work to rotate the cell, so the function of escaping the cell from the ejection orifice will be reduced, and the cell will be captured easily. Here, the length of the ejection orifice unit the distance between the inlet to the outlet of the ejection orifice.

The cell capture unit is not particularly limited, but preferably has a constriction area for capturing cells. The constriction area facilitates cell capture. It is preferable that the width of the constriction area should be narrower than the diameter of the cell to increase the certainty of capture. Since the cell is captured upstream of the constriction area, it is preferable that the constriction area is located downstream of the ejection orifice. Preferably, the cell capture unit comprises a constriction area for capturing cells, and further a detour flow path that does not capture cells. If cells are captured, it becomes difficult for the liquid to flow, causing damage to the cell and causing the liquid to easily overflow from the ejection orifice. If there is a detour flow path that does not capture the cells, these problems are less likely because the liquid can still flow through the detour flow path after capturing the cells.

The cell capture unit is not particularly limited, but it is preferable to capture cells electrically. It is known that cells have different dielectric constants from liquids, and for example, cells can be captured by dielectrophoresis.

The present disclosure also provides a cell ejection method characterized in that the cell is ejected by the above-described cell ejection apparatus. The cell ejection apparatus of the present disclosure can be used arbitrarily in various applications without any particular limitation. Among them, it is suitable for cell dispensing, in which a fixed amount of cells is divided, and for bioprinting, in which cells are arranged in two or three dimensions. The cell ejection apparatus is also suitable for an apparatus and a method for producing cultured meat and an apparatus and a method for producing a tissue, an organ, or a viscus, or the like for regenerative medicine. Specifically, the apparatus for producing the cultured meat comprises the cell culture apparatus, and ejects droplets containing the cells from the cell ejection apparatus onto a substrate to produce the cultured meat formed by the cells. The method for producing cultured meat is a method for producing the cultured meat formed by the cells by using the cell ejection apparatus described above. And the apparatus for producing a tissue, an organ, or a viscus, is an apparatus comprising the cell ejection apparatus, wherein droplets of a cell suspension containing the cells and the liquid are ejected from the cell ejection apparatus onto a substrate to produce a tissue, an organ, or a viscus formed by the cells. The method for producing a tissue, an organ, or a viscus is a method, wherein the cell ejection apparatus produces a tissue, an organ, or a viscus formed by the cells.

### <Cell Suspension>

The cell suspension contains cells and a liquid, wherein the cells are suspended in the liquid.

### (Cell Type)

The cells described herein are not particularly limited, such as adherent cells, floating cells, and spheroids which are aggregates of these cells. The cells can be cell lines or primary cells. The cells can be eukaryotic or prokaryotic, such as mammalian cells, insect cells, plant cells, yeast cells, and Escherichia coli.

### (Cell Diameter)

The diameter of the cells in the cell suspension can be measured by placing the cell suspension on a hemocytometer or the like and using an optical microscope or the like equipped with an image sensor. Using images recorded with an image sensor, the cell diameter can be determined for the purpose based on the distance information corresponding to the previously stored image. After focusing on the cells, it is preferable to record the image and measure the length. If the cell diameter varies, the number average is used as the cell diameter.

### (Liquid)

Liquids are not particularly limited, but include, for example, water and physiological saline. Also exemplified are buffers such as phosphate buffer (hereinafter abbreviated as “PBS”) and Tris. Various media are also exemplified, such as Dulbecco's Modified Eagle Medium (hereinafter abbreviated as “D-MEM”), Iscove's Modified Dulbecco's Medium (hereinafter abbreviated as “IMDM”), Hanks' Balanced Salt Solutions (hereinafter abbreviated as “HBSS”), Minimun Essential Medium-Eagle Earle's Salts Base with Non-Essential Amino Acid (hereinafter abbreviated as “MEM-NEAA”), RPMI (Roswell Park Memorial Institute Medium) 1640, F-12, and the like. Examples also include serum, commercially available electroporation buffers, commercially available FACS analysis buffers, and the like, or infusions such as lactated Ringer's solution. Two or more of these liquids may be mixed and used. The water is preferably deionized by ion exchange or other unit and sterilized by heating in an autoclave or other unit.

Various substances other than cells and liquid may be added to the cell suspension according to the purpose. Examples include, but are not limited to, scaffolding materials, extracellular matrix, growth factors, hormones, amino acids, serum, enzymes, other proteins, DNA, RNA, gelling agents, photocurable resins, labeling agents, salts, sugars, ribonucleotides, pH regulators, pH buffers, surfactants, chelators, organic solvents, antibacterial agents, moisturizers, thickeners, and the like.

### First Embodiment

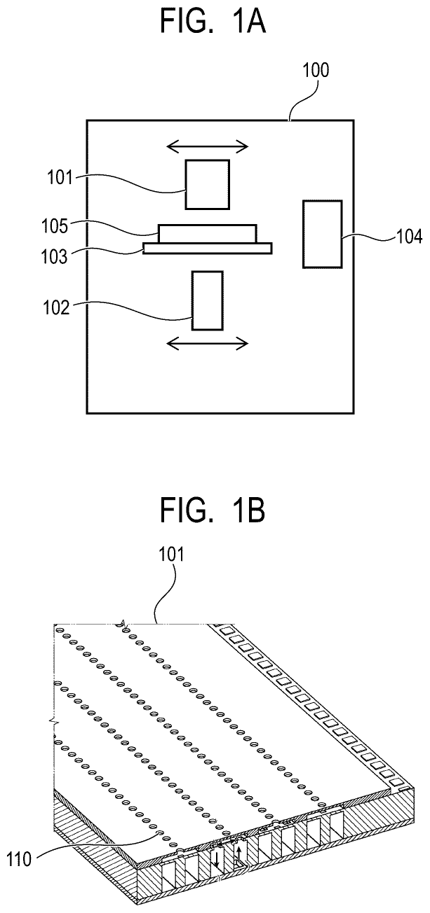

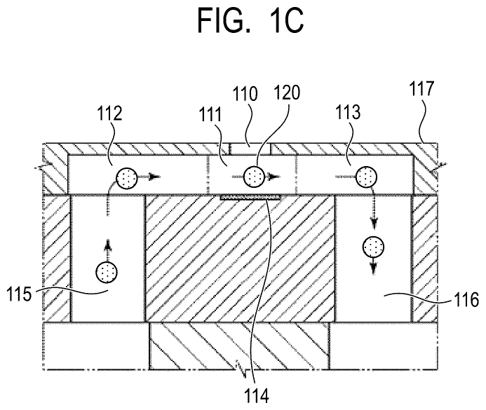

FIGS. 1A, 1B, and 1C are schematic views showing an example of a cell ejection apparatus to which the present disclosure is applied. FIG. 1B is an enlarged view of the cell ejection head 101 of FIG. 1A viewed obliquely upward, and FIG. 1C is a cross-sectional view of a portion including one ejection orifice 110 in the cell ejection head 101 of FIG. 1B. As shown in FIG. 1A, the present cell ejection apparatus 100 comprises an ink jet cell ejection head 101, a microscope 102, a stage 103, a control unit 104, and a dish 105. As shown in FIGS. 1B and 1C, cell ejection head 101 is composed of a liquid chamber 111, an ejection orifice 110 for ejecting a liquid from the liquid chamber 111, a supply flow path 112 for supplying a cell suspension to the liquid chamber 111, and a collection flow path 113 for collecting a liquid or a cell suspension from the liquid chamber 111, which are formed by accumulating a large number of them at intervals of 600 dpi. A plurality of supply flow paths 112 are connected to a common flow path 115 for supply, and a plurality of collection flow paths 113 are connected to a common flow path 116 for collection, separately. In the thermal ink jet system, a heating element 114 as a cell ejection unit is provided in the liquid chamber 111, and when the heating element is electric heated, liquid forms bubbles and droplets are ejected. The cell ejection head 101 is filled with a cell suspension, and a flow from the supply flow path 112 to the collection flow path 113 is generated in the liquid chamber 111 by a pump (not shown). The direction in which the cell suspension flows in the liquid chamber 111 and the direction in which the droplet is ejected from the ejection orifice 110 are substantially perpendicular. The plurality of ejection orifices 110 are arranged in parallel with respect to the flow direction of the cell suspension.

The width of the liquid chamber 111 is 30 μm, which is equal to or less than 5 times the cell diameter, and the height of the liquid chamber 111 is 15 μm, which is equal to or greater than the cell diameter. The length of the ejection orifice 110 is 5 μm, the diameter of the inlet of the ejection orifice 110 is 22 μm, and the diameter of the outlet of the ejection orifice 110 is 20 μm, which is narrowed from upstream to downstream. The volume of the ejection droplet is 5 pl, which is equal to or less than 6.25 times the cell volume. The flow velocity in the flow path is 100 mm/s, and the flow rate is 45 nl/s. The drive frequency of the cell ejection unit that ejects ink jet droplets is 10 Hz.

The cell suspension consists of CHO-K1 cells, which are Chinese hamster ovary cells, suspended in PBS, a phosphate-buffered saline solution. The cell diameter is 12 μm and the volume is 0.9 pl. The cell concentration in the cell suspension is 1×10⁶cells/ml in number concentration and 0.09% in volume concentration. The flow rate× cell number concentration=45, which is larger than the ink jet drive frequency of 10 Hz.

The orifice plate 117 constituting the top plate of the supply flow path 112, the liquid chamber 111 and the collection flow path 113 is formed of an optically transparent resin. A hole is provided in the stage 103, and the transparent dish 105 serving as a medium to be ejected is provided. The microscope 102 is provided below the stage 103 to observe the flow of cell 120 through the dish 105 and the orifice plate 117. At the timing when the cell 120 passes through the liquid chamber 111, the heating element 114 is energized to eject droplets containing the cell 120.

The control unit 104 is connected to the cell ejection apparatus 100 and a personal computer which is an information terminal (not shown), and controls the operation of each part of the cell ejection apparatus 100 in an integrated manner. The cell ejection head 101 and the microscope 102 are operated with respect to the stage 103 without changing the relative positional relationship. When image information is received from the personal computer, an image signal is generated. Also, the allowable deviation amount input to the personal computer is received. Further, image data from the microscope 102 is analyzed to obtain information on the position of the cell 120. Based on these information, the operation of the cell ejection head 101 and the microscope 102, and the ejection at each ejection orifice 110 are controlled.

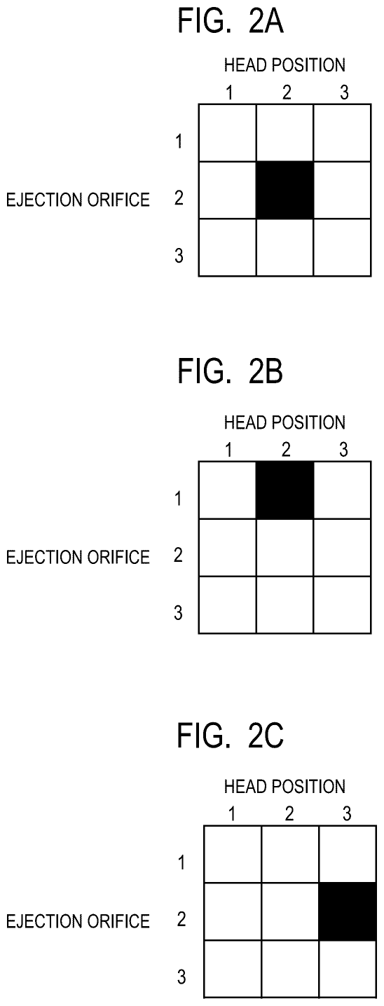

As a simple example, a case in which a pattern is formed from image signals as shown in FIGS. 2A, 2B, and 2C will be described here. The allowable deviation amount is set to 1.

When the cell ejection head position is at 2, it is assumed that a cell passes into the liquid chamber of the ejection orifice 2. In this case, the heating element of the ejection orifice 2 is energized to eject droplets containing the cell from the ejection orifice 2 (FIG. 2A).

When the cell ejection head position is at 2, it is assumed that a cell does not pass through the liquid chamber of the ejection orifice 2 but passes through the liquid chamber of the ejection orifice 1. In this case, since the ejection orifice 1 is in the relationship of the deviation amount 1with the ejection orifice 2, and the deviation amount is within the allowable deviation amount 1, the heating element of the ejection orifice 1 is energized to eject droplets containing the cell from the ejection orifice 1 (FIG. 2B).

When the cell ejection head position is at 2, the cell does not pass through the liquid chambers of the discharge ports 1, 2 and 3, and when the cell ejection head position is at 3, the cell passes through the liquid chamber of the ejection orifice 2. In this case, since the cell ejection head position 3 is in the relationship of the deviation amount 1 with the cell ejection head position 1, and the deviation amount is within the allowable deviation amount 1, the heating element of the ejection orifice 2 is energized at the cell ejection head position 3 to eject droplets containing the cell from the ejection orifice 2 (FIG. 2C).

By controlling the ejection timing and the ejection orifice to be ejected on the basis of the information on the position of the cells and the image information, it is possible to efficiently eject the droplets containing cells while reducing the ejection of the droplets not containing cells. In the absence of the allowable deviation amount, the cell ejection head cannot be moved until the required ejection is performed at each ejection orifice, but the allowable deviation amount makes it possible to eject droplets containing cells while moving the cell ejection head.

### Second Embodiment

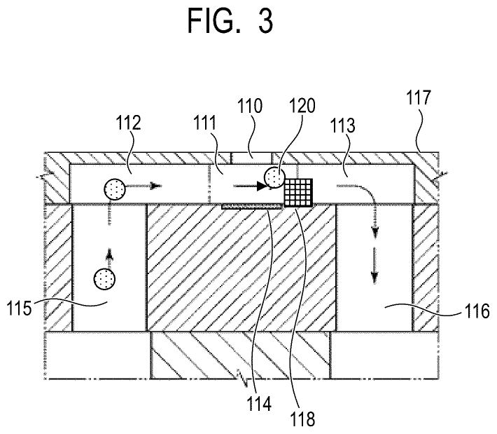

FIG. 3 is a schematic view showing another example of a cell ejection apparatus to which the present disclosure is applied. The cell ejection apparatus of this embodiment does not have the microscope 102 provided in the first embodiment, and does not detect the position of the cell 120. Since the position of the cell 120 is not detected, the ejection control based on the information on the position of the cell 120 is also not performed. Instead, there is a constriction area from the liquid chamber 111 to the collection flow path 113.

The height of the member 118 for forming the constriction area (cell capture unit) is 10 μm, and its length is 10 μm. The constriction area has a flow path height of 5 μm, which is equal to or less than the cell diameter. The drive frequency of the cell ejection unit for ejecting the droplets by an ink jet system is 45 Hz. Other configurations and conditions are the same as those of the first embodiment.

The cell 120 in the cell suspension is captured upstream of the constriction area. Flow rate×number concentration of cells=45, which is equal to the ink jet drive frequency of 45 Hz. One cell 120 is captured on average during the driving cycle. In this state, the heating element 114 is energized to eject droplets containing the cell 120.

### Third Embodiment

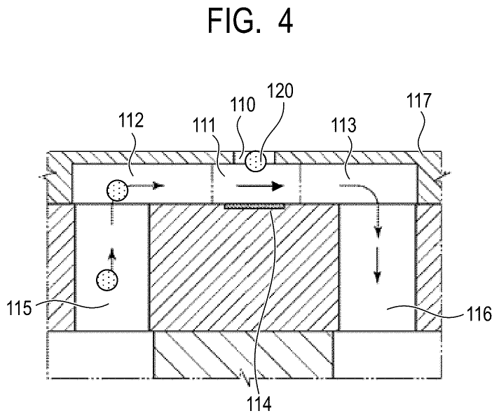

FIG. 4 is a schematic view showing another example of a cell ejection apparatus to which the present disclosure is applied. The cell ejection apparatus according to the present embodiment does not have the constriction area provided in the second embodiment, and the ejection orifice 110 serves also as a cell capture unit. The length of the ejection orifice 110 is 9 μm, which is equal to or greater than the cell radius, and equal to or less than the cell diameter. Other configurations and conditions are the same as those of the second embodiment.

The cell 120 passes through the ejection orifice 110, and once the cell is captured in the ejection orifice, a flow that cannot be escaped occurs. Therefore, the cell 120 is captured in the ejection orifice 110. In this state, the heating element 114 is energized to eject droplets containing the cell 120.

The present disclosure is not limited to the embodiments described above, and various changes and modifications may be made without departing from the spirit and scope of the present disclosure. Accordingly, the following claims are appended hereto in order to make the scope of the present disclosure public.

According to the present disclosure, it is possible to provide an ink jet cell ejection apparatus which can reduce the time required for ejecting a predetermined amount of droplets of a cell suspension containing a cell.

While the present disclosure has been described with reference to embodiments, it is to be understood that the present disclosure is not limited to the disclosed embodiments. The scope of the following claims is to be accorded the broadest interpretation so as to encompass all such modifications and equivalent structures and functions.

1. A cell ejection apparatus for ejecting a droplet of a cell suspension containing a cell and a liquid by an ink jet system,
wherein the cell ejection apparatus comprises
a liquid chamber holding the cell suspension,
an ejection orifice for ejecting the droplet from the liquid chamber,
a supply flow path for supplying the cell suspension to the liquid chamber,
a collection flow path for collecting the liquid or the cell suspension from the liquid chamber,
and a cell ejection unit for selectively ejecting the droplet from the ejection orifice so that the droplet contains the cell.

2. The cell ejection apparatus according to claim 1, wherein the cell ejection apparatus has a plurality of the ejection orifices, and the plurality of the ejection orifices are integrally formed.

3. The cell ejection apparatus according to claim 1, wherein a direction in which the cell suspension flows in the liquid chamber and a direction in which the droplet is ejected from the ejection orifice are substantially perpendicular.

4. The cell ejection apparatus according to claim 3, wherein the liquid chamber has a maximum width W1 and a minimum width W2 in a direction perpendicular to the direction in which the cell suspension flows, and satisfies
D≤W2≤W1≤5D
when the diameter of the cell is D.

5. The cell ejection apparatus according to claim 1, wherein the ink jet system is a thermal ink jet system driven by bubble formation of the liquid as heated.

6. The cell ejection apparatus according to claim 1, wherein the supply flow path and the collection flow path are connected to different common flow paths.

7. The cell ejection apparatus according to claim 1, wherein a flow rate Q (ml/s) of the cell suspension flowing through the liquid chamber, a number concentration of the cells p (cells/ml) in the cell suspension, and a drive frequency f (Hz) of the cell ejection unit for ejecting the droplet in an ink jet system satisfy
Q×p≥f.

8. The cell ejection apparatus according to claim 1, wherein a volume of the droplet to be ejected is 100 times or less of the cell volume.

9. The cell ejection apparatus according to claim 8, wherein the volume of the droplet to be ejected is 6.25 times or less of the cell volume.

10. The cell ejection apparatus according to claim 1, wherein the ejection orifice is formed to narrow from upstream to downstream in the ejection direction of the droplet.

11. The cell ejection apparatus according to claim 1, wherein the cell ejection unit comprises:
at least one of a cell detection unit for detecting a position of the cell in the cell suspension and a cell supply control unit for controlling the position of the cell in the cell suspension, and an ejection control unit for controlling at least one of a timing for ejecting the droplet and a location of the ejection orifice for ejecting the droplet, based on at least one of information on the position of the cell in the cell suspension obtained by the cell detection unit and information on the position of the cell in the cell suspension controlled by the cell supply control unit.

12. The cell ejection apparatus according to claim 11, wherein the cell detection unit optically acquires information on the position of the cell in the cell suspension.

13. The cell ejection apparatus according to claim 12, wherein at least one of the liquid chambers, the supply flow path, and the collection flow path is at least partially optically transparent.

14. The cell ejection apparatus according to claim 11, wherein the cell detection unit electrically acquires information on the position of the cell in the cell suspension.

15. The cell ejection apparatus according to claim 14, wherein the cell detection unit acquires information on the position of the cell in the cell suspension by measuring impedance.

16. The cell ejection apparatus according to claim 11, wherein the ejection control unit controls ejection based on both the information on the position of the cell in the cell suspension and furthermore an image signal.

17. The cell ejection apparatus according to claim 16, wherein the ejection control unit controls ejection based on the information on the position of the cell in the cell suspension, the image signal, and furthermore an allowable deviation amount allowed between the information on the position of the cell in the cell suspension and the image signal.

18. The cell ejection apparatus according to claim 1, wherein the cell ejection unit comprises a cell capture unit for capturing the cells in the cell suspension.

19. The cell ejection apparatus according to claim 18, wherein the cell capture unit is provided in at least one of a liquid chamber, a supply flow path, and a collection flow path.

20. The cell ejection apparatus according to claim 11, wherein the ejection orifice also serves as a cell capture unit.

21. The cell ejection apparatus according to claim 20, wherein the length of the ejection orifice is not less than the radius of the cell, and not more than 5 times the diameter of the cell.

22. The cell ejection apparatus according to claim 20, wherein the length of the ejection orifice is not less than the radius of the cell, and not more than the diameter of the cell.

23. The cell ejection apparatus according to claim 18, wherein the cell capture unit comprises a constriction area for capturing the cell.

24. The cell ejection apparatus according to claim 23, wherein the cell capture unit further comprises a bypass flow path that does not capture the cell.

25. The cell ejection apparatus according to claim 18, wherein the cell capture unit electrically captures the cell.

26. The cell ejection apparatus according to claim 1, wherein the cell ejection apparatus is an apparatus for use in cell dispensing.

27. The cell ejection apparatus according to claim 1, wherein the cell ejection apparatus is an apparatus for use in bioprinting to arrange the cell in two or three dimensions.

28. A cell ejection method, wherein the cell is ejected by the cell ejection apparatus according to claim 1.

29. An apparatus for producing cultured meat, comprising the cell ejection apparatus according to claim 1,
wherein a droplet of a cell suspension containing the cell and the liquid is ejected from the cell ejection apparatus onto a substrate to produce cultured meat formed by the cells.

30. A method for producing cultured meat, wherein the cultured meat formed by the cells is produced by the cell ejection apparatus according to claim 1.

31. An apparatus for producing a tissue, an organ, or a viscus,
comprising the cell ejection apparatus according to claim 1,
wherein a droplet of a cell suspension containing the cell and the liquid is ejected from the cell ejection apparatus onto a substrate to produce a tissue, an organ, or a viscus formed by the cells.

32. A method for producing a tissue, an organ, or a viscus, wherein the cell ejection apparatus according to claim 1 produces a tissue, an organ, or a viscus formed by the cells.
